(19) United States
(12) Patent Application Publication
(10) Pub. No.: US 2014/0350427 A1
(43) Pub. Date: Nov. 27, 2014

(54) SUPPLEMENTAL GAS DELIVERY AND MONITORING SYSTEM

(71) Applicant: KONINKLIJKE PHILIPS N.V., EINDHOVEN (NL)

(72) Inventor: Gary Nathan Holder, Ringgold, GA (US)

(21) Appl. No.: 14/345,438

(22) PCT Filed: Sep. 21, 2012
(86) PCT No.: PCT/IB2012/055013

§ 371 Date: Mar. 18, 2014

Related U.S. Application Data
(60) Provisional application No. 61/537,735, filed on Sep. 22, 2011.

(51) Int. Cl. A61M 16/00; A61M 16/10; A61B 7/04; A61B 7/00; A61B 5/00; A61M 16/06; A61B 5/08

(52) U.S. Cl. CPC A61M 16/0051; A61M 16/0672; A61M 16/1005; A61M 16/0069; A61B 5/0823; A61B 7/003; A61B 5/7405; A61B 5/742; A61B 5/746; A61B 5/747; A61B 5/0022; A61B 7/04; A61B 5/6819; A61B 5/4839; A61M 16/0003
USPC 600/529; 128/202.22

(57) ABSTRACT

An method and system for supplemental gas delivery that enables the identification of a need of therapeutic attention for patient receiving the supplemental gas (20) by detecting an onset of an exacerbation of a medical condition. Such a detection is performed by detecting audible events using a microphone (60) that is in fluid communication with a cannula (28) that is used to communicate the supplemental gas (20) with the airways of the patient (8). The audible event may be, for instance, a cough in the patient. A change in the relative frequency of coughs in the patient may be identified by the routines as being indicative of an onset of an exacerbation.

### CROSS-REFERENCE TO RELATED APPLICATIONS

This patent application claims the priority benefit under 35 U.S.C. §119(e) of U.S. Provisional Application No. 61/537,735 filed on Sep. 22, 2011, the contents of which are herein incorporated by reference.

### BACKGROUND OF THE INVENTION

1. Field of the Invention

The present invention pertains to delivery of a supplemental gas to patient, and, in particular, to an improved system and method of indicating a need of therapeutic attention for a patient receiving the supplemental gas.

2. Description of the Related Art

There are numerous situations where it is necessary or desirable to deliver a flow of supplemental gas, such as oxygen, to a patient. For example, the delivery of supplemental oxygen is widely used for the long-term treatment of chronically ill patients suffering from various respiratory diseases such as Chronic Obstructive Pulmonary Disease (COPD) and emphysema. Additionally, in emergency situations, supplemental oxygen is administered on a short-term basis to relieve acute symptoms, such as shortness of breath and lowered oxygen saturation. Supplemental oxygen is also commonly administered throughout the hospital setting, such as in the operating room during surgery and post-op, and in the intensive care units to critically ill patients.

In patients suffering from COPD, a flow of essentially pure oxygen is provided on a generally constant basis because the pulmonary system of a long-term oxygen patient with COPD is typically in a state of progressive failure. That is, the patient's pulmonary system is unable to provide sufficient oxygen for use by the patient's organs, etc., merely by the breathing of air. Because the condition of such a patient is almost always terminal, it is desirable to avoid any exacerbation of the condition or, if such an exacerbation is discovered, to alleviate it as soon as possible. The alleviation of such an exacerbation typically requires an extended and costly hospital stay which would preferably be avoided.

However, an exacerbation of a COPD medical condition can sometimes be evidenced by symptoms that are sufficiently minor or hard to detect that the patient does not actually realize that such an exacerbation is underway. Moreover, the patient may be in a state of denial that an exacerbation is underway.

### SUMMARY OF THE INVENTION

Accordingly, it is an object of the present invention to provide a supplemental gas delivery system and method that overcomes the shortcomings of conventional systems. This object is achieved according to one embodiment of the present invention by providing an improved method and oxygen supply system that enables the identification of a need of therapeutic attention for a long-term oxygen patient. This is accomplished by detecting an onset of an exacerbation of a medical condition, such as COPD. Such a detection is performed monitoring audible events using the same cannula that is in fluid communication with the airways of the patient to deliver the supplemental gas, such as oxygen. The audible events may be, for instance, indicative of coughs in the patient. A change in the relative frequency of coughs in the patient may be identified by the routines as being indicative of an onset of an exacerbation. Advantageously, therefore, the method and system provide an output that indicates a need of therapeutic attention for the patient.

Accordingly, an object of the present invention is to provide an improved method of detecting an onset of an exacerbation of COPD or other medical condition in a patient and of indicating a need of therapeutic attention for the patient.

Another object of the present invention is to provide an improved method and system that can provide to any of a number of individuals such as caretakers, medical professionals, and the actual patient, an indication of the need of therapeutic attention for the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

In certain embodiments, the general nature of the invention can be stated as including a method of indicating a need of therapeutic attention for a long-term oxygen patient. The general nature of the method can be stated as including providing a flow of a supplemental gas, such as oxygen, to a patient through a cannula in fluid communication with the airway of the patient, detecting within at least a portion of the cannula audible events, processing the audible events to determine whether the patient is experiencing an onset of an exacerbation of a medical condition, and, responsive to the determination, providing an output indicative of a need of therapeutic attention for the patient.

In certain embodiments, the general nature of the invention can be stated as including a supplemental gas supply system that is structured to provide a flow of gas to a patient. The system can be stated as including a processing system that includes a processor and a storage, a gas source, a cannula in fluid communication between the gas source and an airway of the patient, and a sensor apparatus that includes a pressure sensor and a microphone and is in fluid communication with at least a portion of the cannula, with the sensor apparatus being connected with the processor apparatus. The processor receives the output from the pressure sensor and microphone and detects audible events based on the output from at least the microphone. The processor then determines whether the patient is experiencing an onset of an exacerbation of a medical condition based on the monitored audible events and, responsive to the determination, provides an output indicative of a need of therapeutic attention for the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention

### BRIEF DESCRIPTION OF THE DRAWINGS

FIG. 1 is a schematic depiction of an improved oxygen supply system in accordance with the invention connected with a long-term oxygen patient;

FIG. 2 is a diagrammatic depiction of a portion of the improved oxygen supply system of FIGS. 1; and

FIG. 3 is flowchart depicting an improved method in accordance with the invention which can be performed with the system of FIG. 1.

### DETAILED DESCRIPTION OF EXEMPLARY EMBODIMENT

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

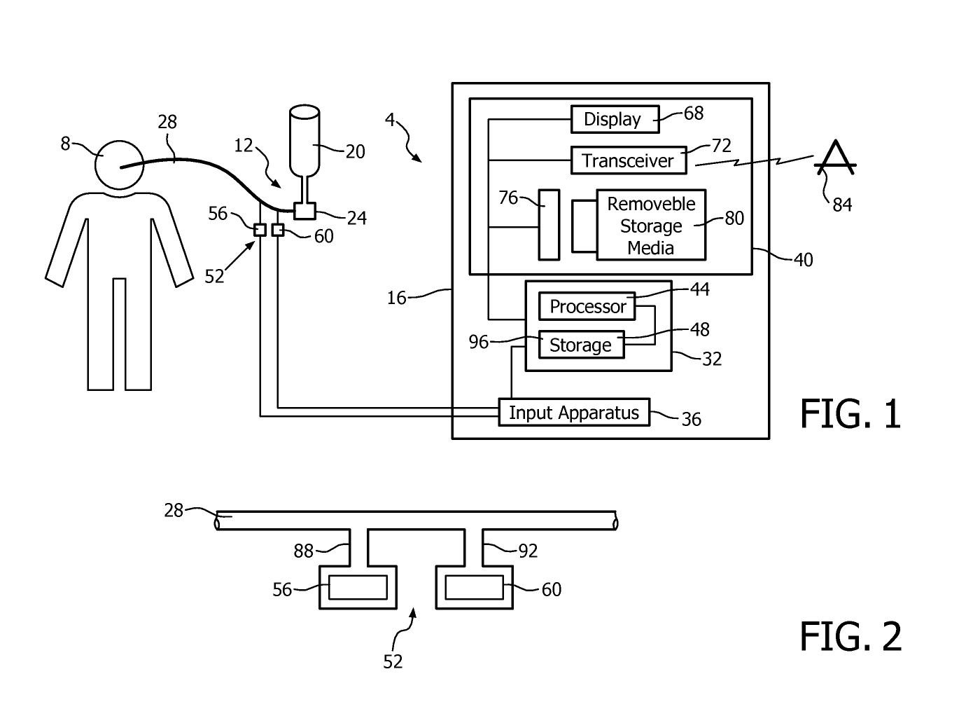

An improved supplemental gas supply system 4 in accordance with the present invention is depicted in FIG. 1 as being connected in fluid communication with a long-term oxygen patient 8 who has a medical condition such as COPD or other medical condition. As is understood in the relevant art, gas supply system 4 is configured to provide a therapeutic flow of breathing gases, such as breathing oxygen to patient 8. It is to be understood that any supplemental gas or combinations of gases, e.g., heliox, are contemplated as being suitable for use in the present invention.

Supplemental gas supply system 4 can be broadly construed as including an oxygen unit 12 and a control unit 16 that are connected together. Oxygen unit 12 can be stated to include an oxygen source 20, a valve 24, and a cannula 28, and these elements are in fluid communication with one another. Cannula 28 is, in the depicted exemplary embodiment, a single cannula 28 that is connected in fluid communication between valve 24 and the nostrils of patient 8, although multiple cannula can be employed depending upon the needs of the particular application.

Oxygen source 20 can be any of a wide variety of sources of breathable oxygen, such as an oxygen tank or receptacle, an oxygen concentrator, and the like without limitation. In the illustrated embodiment, oxygen source 20 is in fluid communication with valve 24 which controls the delivery of breathing oxygen to cannula 28 and thus to patient 8. Control unit 16 typically is in operative control of valve 24, although this need not necessarily be the case. The present invention contemplates using any conventional technique for controlling the flow of the supplemental gas to the patient.

Control unit 16 can be said to include a processing system 32, an input apparatus 36, and an output apparatus 40 that are electronically connected together. Processing system 32 can be said to include a processor 44 and a storage 48 in communication with one another. Processor 44 can be any of a wide variety of processors such as microprocessors and the like without limitation. Storage 48 can be any of a wide variety of storage devices that can interface with processor 44 and which can include, for example and without limitation, RAM, ROM, EPROM, EEPROM, FLASH, etc., on which a number of routines or data or both can be stored for processing by processor 44.

Input apparatus 13 is structured to provide input to processing system 32 and in particular includes a sensor apparatus 52 that includes a pressure sensor 56 and a microphone 60. Input apparatus 36 may additionally include other input devices such as a keypad, touchpad, mouse, etc., and may also include the touch-sensitive component of a touch-sensitive display.

As is understood in the relevant art, pressure sensor 56 and microphone 60 are both sensitive to variations in the pressure of a fluid, such as air. It is noted, however, that pressure sensor 56 is of a type that is configured to detect an instantaneous or static pressure differential, such as between a detected pressure and a reference pressure. In contrast, microphone 60 is of a type that detects vibrations within a fluid, with the vibrations being characterized by changes in pressure as a function of time. Such vibrations may be audible and may be in the form of periodic pressure changes in the fluid, whether or not transient.

Output apparatus 40 can include any of a wide variety of devices that receive output signals from processing system 32 and which provide output in any of a variety of forms that are receivable or detectable by other devices, including persons. The exemplary output apparatus 40 depicted herein includes a visual display 68, a wireless transceiver 72, and an output port 76 that is connected with a removable array of storage media 80. Display 68 can be, for instance, a display such as an LCD display or other type of display, and may include the visual component of a touch-sensitive display of the type suggested above. Alternatively, display 68 may be in the form of one or more warning light that can be triggered to be illuminated or to flash depending upon instructions generated by processing system 32.

Wireless transceiver 72 can be any of a wide variety of transceivers that transmit and/or receive electromagnetic signals with respect to a remote device 84. For example and without limitation, wireless transceiver 72 may be in the nature of a Wi-Fi transceiver that is wirelessly connectable with remote device 84 when remote device 84 is in the form of a wireless router, wireless access point, etc., such as might employ any of the IEEE 802.11 communication protocols and the like. Alternatively, wireless transceiver 72 may be in the form of a cellular transceiver which would communicate with remote device 84 when remote device 84 is in the form of a cellular tower connected with a telephone transmission network. Still alternatively, wireless transceiver 72 may be replaced with a wired connection device such as one that may include a plug for connection with an RJ-45 cable or other connector that is connected with remote device 84 when remote device 84 is in the form of a router or access point having a connection with the Internet.

Output port 76 can be any of a wide variety of electronic communication ports that enable removable storage media 80 to be electronically connected with processing system 32. Output port 76 thus could be, for instance and without limitation, a Universal Serial Bus (USB) port when storage media 80 is in the form of a USB storage device such as a FLASH memory device, etc. Other known types of removable storage media can be employed for storage media 80, with output port 76 thus being configured to cooperate with storage media 80. It is particularly noted that storage media 80 is removable from output port 76 in order that it can be transported and delivered to another device or can be provided to a person such as a medical professional, for example and without limitation.

As can be understood from FIG. 2, pressure sensor 56 is connected in fluid communication with cannula 28 via a first fluid connection 88. Similarly, microphone 60 is connected in fluid communication with cannula 28 via a second fluid connection 92. First and second fluid connections 88 and 92 are in the exemplary form of T-connections with cannula 28 in the vicinity of the connection of cannula 28 with valve 24, although other arrangements and positioning can be employed without departing from the present concept. As is depicted schematically in FIG. 1 (but is not depicted in FIG. 2 for purposes of clarity), pressure sensor 56 and microphone 60 each have a wired output that is electronically connected with processing system 32. By placing pressure sensor 56 and microphone 60 in fluid communication with cannula 28, pressure sensor 56 can sense within the interior of cannula 28 a pressure that is in comparison with a reference pressure such as ambient pressure, and microphone 60 can detect vibrations such as may include audible signals within the interior of cannula 28. Pressure sensor 56 and microphone 60 can communicate such signals via their wires to processing system 32 for processing thereby.

It is noted that processing system 32 includes one or more processing routines that are collectively referred to herein with the numeral 96 and that are stored in storage 48 and which are executable on processor 44 in order to cause oxygen supply system 4 to perform certain advantageous operations. Routines 96 may include, by way of example and without limitation, a graphical user interface (GUI) routine 96 and a breathing detection routine 96. Routines 96 may also include a sound recognition routine 96 that potentially may include a learning component and that potentially may also include one or more signature signals that can be used for comparison with signals detected in the interior of cannula 28 by sensor apparatus 52 to determine, for instance, the nature of an audible event.

The breathing detection routine 96 may employ variations in the air pressure within cannula 28 in order to determine the occurrences of exhalation and inhalation, with the flow of oxygen to patient 8 being reduced or curtailed during exhalation events via control of valve 24, by way of example. The breathing detection routine 96 may additionally or alternatively be used to determine from the duration and timing between exhalation and inhalation events to determine whether patient 8 is sleeping and, if so, to changes the flow/pressure of the oxygen within cannula 28 by controlled operation of valve 24, again by way of example. Other uses of the breathing detection routine 96 can be envisioned. The change in pressure can be any appropriate change, such as an increase or a decrease in the flow/pressure.

The sound recognition routine 96 may employ signals from microphone 60, potentially with the addition of signals from pressure sensor 56, to determine the cause of certain audible events or occurrences that are detected within cannula 28. An example of such an audible event may be a series of sounds that are produced in a transient fashion in response to a cough by patient 8. For example, microphone 60 may generate electronic signals representative of frequencies of vibrations of the air within cannula 28 that can occur when patient 8 coughs. Possibly additionally, pressure sensor 56 may generate electronic signals representative of fluid pressures within cannula 28, with the fluid pressures changing in accordance with a particular transient characteristic indicative of a cough in patient 8.

The sound recognition routine 96 may include one or more signature signals that are representative of the audible content of a cough, such as a cough by patient 8 or a generic cough signature signal, with such signature signals being compared with the sound signals generated by microphone 60 in order for the sound recognition routine 96 to determine whether an audible event was due to a cough by patient 8 as compared with, say, a sneeze or a clearing of the throat by patient 8. The signature signals potentially could be generated by patient 8 during the initial setup of oxygen supply system 4, such as by instructing patient 8 to cough a predetermined number of times with such coughs being recorded and used to generate such signature signals, or the signature signals could potentially be of a more generic type that are generated and stored in advance of oxygen supply system 4 being connected with patient 8. Furthermore, the sound recognition routine 96 potentially may employ a learning component that can be used to adjust its signature signals or to adjust its own algorithms based upon repeated occurrences of audible events that are determined by the sound recognition system 96 to be coughs by patient 8. Other variations are possible within the scope of the present invention.

It is particularly noted that an exacerbation detection routine 96 is also stored in storage 48 for execution on processor 44 and that is configured to employ output such as those from the breathing detection routine 96 or the sound recognition routine 96 or both to detect an onset of an exacerbation of a medical condition such as COPD or other medical condition. For example, the exacerbation routine 96 may detect an increase in the frequency over time of coughs by patient 8. That is, the exacerbation detection routine 96 might determine that patient 8 typically coughs fourteen times per week, i.e., twice a day on average, and may use such a determination as a benchmark as part of the determination of whether the COPD of patient 8 is becoming exacerbated.

The exacerbation detection routine 96 in such a situation may determine, by way of example, that over a period of five consecutive days patient 8 coughed three times per day on the first, third, and fifth days, but coughed only twice per day on the second and fourth days. Such an increase in coughing by patient 8 may be insignificant enough in magnitude that patient 8 may not even notice the slight increase in coughing, but the exacerbation detection routine 96 may be configured to recognize that such an increase in the frequency of coughing is sufficiently indicative of an onset of an exacerbation of the COPD that the processing system 32 may generate an output signal for output apparatus 40 indicating that patient 8 is experiencing an exacerbation of a medical condition.

As suggested above, such an output can be something that can be perceived by patient 8 or a caretaker of patient 8 or both, and/or the output can be generated for delivery to a medical professional. Such an output can be delivered directly to such a medical professional via wireless transceiver 72 in communication with remote device 84 and, for instance, some type of a network, or can be stored in removable storage media 8 that may be periodically removed and provided to the medical professional. It is noted, however, that the exacerbation detection routine 96 may employ any of a wide variety of criteria in determining whether or not an exacerbation of a medical condition such as COPD is beginning to occur or already has occurred. In this regard, it is also noted that coughs are presented herein merely as an example of an incident that can produce an audible result which can be employed in determining an onset of an exacerbation, it being further noted that other audible events and other events may be additionally or alternatively employed without departing from the scope of the present invention.

By detecting an onset or occurrence of an exacerbation of a condition and responsively providing an output that is indicative of a need of therapeutic attention for patient 8, oxygen supply system 4 can advantageously signal the need for therapeutic attention well before patient 8 is in need of an extended hospital stay to alleviate the exacerbation. This has the advantageous effect of not only reducing cost but also improving the quality of life of patient 8 and enabling the more rapid provision of therapeutic medical care to patient 8. Other advantages will be apparent.

In addition to providing an output that is indicative of a need of therapeutic attention for patient 8, oxygen supply system 4 can also advantageously responsively increase (or decrease) the oxygen dosage slightly to possibly help patient 8. Additionally or alternatively, oxygen supply system 4 can recommend to patient 8 that they increase (or decrease) their breathing treatment or some other medical treatment. This feature could be pre-approved by the medical professional. For example, a doctor could program oxygen supply system 4 to indicate to patient 8 a different drug or medical regimen if a cough rate exceeds some predetermined threshold. Oxygen supply system 4 can could also communicate using the output apparatus 40 to a number of other devices such as a CPAP machine, a nebulizer, an automatic pill dispenser, etc. to change the therapy being provided by such devices.

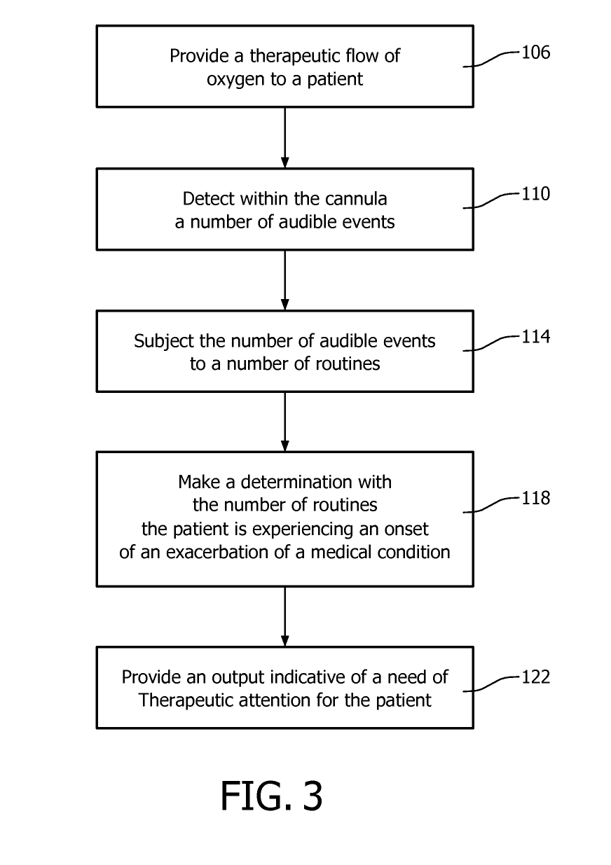

An improved method of indicating a need of therapeutic attention for patient 8 in accordance with the present invention is described with reference to a flowchart depicted generally in FIG. 3. The method can be generally stated as indicating a need of therapeutic attention in patient 8 by providing an output that indicates an onset or an occurrence of an exacerbation of a medical condition, such as an exacerbation of COPD.

The method can be stated as beginning, as at 106, with providing a therapeutic flow of breathing gas, such as oxygen, to patient 8 through cannula 28 which is in fluid communication with the airways of patient 8. Microphone 60 and also possibly pressure sensor 56 detect, as at 110, conditions within the interior of cannula 28 that occur due to audible events, such as coughs by patient 8, as suggested above. In this regard, it is reiterated that the audible events that can be used in detecting an exacerbation of a medical condition are not merely limited to those resulting from coughs and can include, by way of example, those responsive to wheezing instances, crackling instances, sneezing instances, snoring instances, and potentially can also be responsive to intentional audible events generated by patient 8, such as through the use of humming, speaking a predetermined word, and the like.

Processing continues, as at 114, where the various audible events are subjected to processing routines 96, such as those routines 96 mentioned above and others. These routines 96 are employed, as at 118, in making a determination that patient 8 is experiencing an onset of an exacerbation of the medical condition. In this regard, it is noted that the routines 96 likely are of sufficient sophistication that they are able to distinguish between a cough by patient 8 that is detected within the interior of cannula 28 and a cough by another person, such as another patient or other individual, that is sufficiently close to cannula 28 and/or is sufficiently loud that it can be detected by microphone 60 within the interior of cannula 28.

Processing then continues, as at 122, where, responsive to the determination made at 118 that patient 8 is experiencing an exacerbation of the medical condition, an output is provided by output apparatus 40 that is indicative of a need of therapeutic attention or other medical attention for patient 8. As suggested above, the output potentially can be perceived by patient 8 and/or a caretaker of patient 8 and/or can be communicated to a medical professional.

The advantageous oxygen delivery system 4 and method described herein thus save costs and improve patient lifestyle by detecting an onset of an exacerbation of a medical condition and by responsively providing an output indicative of a need of therapeutic attention for patient 8. Other advantages will be apparent to those of ordinary skill in the art.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of indicating a need of therapeutic attention for a patient receiving a flow of supplemental gas, the method comprising:
providing a flow of supplemental gas to a patient through a cannula in fluid communication with an airway of the patient;
detecting within at least a portion of the cannula a plurality of audible events occurring over a period of time that include a current audible event using a microphone operatively coupled to the cannula;
processing the current audible event to determine from the current audible event whether the frequency over time of such audible events has increased and, if so, making a determination that the patient is experiencing an onset of an exacerbation of a medical condition; and
responsive to the determination, providing to a caregiver or to the patient an output indicative of a need of therapeutic attention for the patient.

2. The method of claim 1, further comprising providing as the output at least one of a visible output and an audible output.

3. The method of claim 1, further comprising providing as the output a signal for communication to a medical professional or a domestic caretaker of the patient.

4. The method of claim 3, further comprising storing the signal on a removable storage medium, transmitting the signal over a network, or both storing and transmitting the signal.

5. The method of claim 1, wherein the detecting step includes detecting a cough as the current audible event.

6. The method of claim 5, wherein the processing step includes comparing the current audible event with a signature signal representative of a cough in the patient and, responsive to the comparing, determining that the at least first audible signal represents an occurrence of a cough in the patient.

7-8. (canceled)

9. The method of claim 1, further comprising providing as at least a portion of the output an output recommending, instructing, or providing a change in therapy.

10. Supplemental gas supply system comprising:
a processing system comprising a processor and a storage; a source of supplemental gas;
a cannula in fluid communication between the source and an airway of the patient;
a sensor apparatus operatively coupled to the cannula, the sensor apparatus comprising a pressure sensor, and a microphone both in fluid communication with at least a portion of the cannula, the sensor apparatus being connected with the processor apparatus;
the processing system further comprising a processor adapted to perform operations comprising:
detecting a plurality of audible events occurring over a period of time that include a current audible event based on the output of the microphone;
determining from the current audible event whether the frequency over time of the audible events has increased and, if so, making a determination that the patient is experiencing an onset of an exacerbation of a medical condition based on the current audible event; and
responsive to the determination, providing to a caregiver or to the patient an output indicative of a need of therapeutic attention for the patient.

11. The system of claim 10, wherein the operations further comprise providing as the output a visible output, an audible output, a signal for communication to a medical professional or domestic caregiver, or any combination thereof.

12. The system of claim 10, wherein the operations further comprise providing as the output, a signal and storing the signal on a removable storage medium.

13. The oxygen supply system of claim 10, wherein the operations further comprise providing as the output, a signal and transmitting the signal over a network.

14. The oxygen supply system of claim 10, wherein the operations further comprise detecting a cough by the patient based on the detected current audible event.

15. The oxygen supply system of claim 14, wherein the operations further comprise comparing the current audible event with a signature signal representative of a cough in the patient and, responsive to the comparing, determining that the current audible event represents an occurrence of a cough in the patient.
